(19) United States
(12) Patent Application Publication
(10) Pub. No.: US 2025/0048979 A1
(43) Pub. Date: Feb. 13, 2025

(54) IRRIGATION SYSTEM, IRRIGATION SENSOR AND SMART SCHEDULING FOR IRRIGATION, PROCESSES, AND METHODS OF USE

(71) Applicant: Daniel Zhao, Pasadena, CA (US)

(72) Inventor: Daniel Zhao, Pasadena, CA (US)

(21) Appl. No.: 18/800,071

(22) Filed: Aug. 11, 2024

Related U.S. Application Data
(60) Provisional application No. 63/532,354, filed on Aug. 12, 2023.

(51) Int. Cl. A01G 25/16; G01N 33/24

(52) U.S. Cl. CPC A01G 25/167; G01N 33/245; G01N 33/246

(57) ABSTRACT

An irrigation system, an irrigation sensor system and smart scheduling for irrigation purposes, processes, and methods of use are presented. The irrigation system is configured with a plurality of wireless soil moisture sensors and a plurality of irrigation controllers. The system utilizes a predetermined set of rules and ongoing machine learning to adapt the rules. The system also integrates artificial intelligence and a number of other components to automatically provide irrigation to a desired system which saves water, energy, time, and provides benefits to a user and a global community.

### CROSS-REFERENCE TO RELATED APPLICATIONS

The present application claims priority to the U.S. Provisional Patent Application No. 63/532,354 which was filed on Aug. 12, 2023, which is hereby incorporated by reference herein in its entirety, including any figures, tables, or drawings.

### FIELD OF THE INVENTION

This disclosure relates to an irrigation system, an irrigation sensor system and smart scheduling for irrigation purposes, processes, and methods of use. More specifically, and without limitation, the present disclosure relates to a wireless communication system, a wireless sensor system, and an integrated irrigation controller. More specifically, and without limitation, the present disclosure relates to a plurality of moisture sensors which contribute to irrigation schedules.

### BACKGROUND OF THE INVENTION

Irrigation of plants is well known in the art. Specifically, lawns having grass and the like are well known. Lawns are a common and desirable external feature around homes, businesses, apartment complexes, and the like. Lawns, which contain grass and/or different types of grass require irrigation and/or regular watering in order to thrive and appear a desirable green. Water shortages, water expenses, and water conservation are increasingly important in the presence of population increase, climate change, and the like.

Irrigation controllers are well known in the art. Irrigation controllers are designed and configured to operate an automatic irrigation system, such as lawn and sprinkler systems. Lawn and sprinkler systems are commonly underground pipes that supply water to a lawn or crop at designated time intervals for designated periods of time. Said another way, an irrigation system, or water sprinkler, is a device used to irrigate crops, lawns, and the like. Present day irrigation systems apply water in a controlled manner which is meant to emulate rainfall and/or natural water distribution.

In the present state of the art, modern irrigation systems are configured to run automatically. In the present art, after initial setups and discounting regular maintenance through the different seasons, automatic systems are designed to operate by a central controller which can be configured to run irrigation systems for a specified duration of time. For example, modern irrigation systems will run a particular zone of an irrigation system for 5 minutes, or 10 minutes or another time which is set by a user. These zones may run every night, every other day, or on days as set up by a user.

Current irrigation systems waste water and/or when using water, water by inefficient means which has not kept up with modern understanding of agricultural science. Modern irrigation systems fail to account for rain, recent rainfall, sun time, soil type, soil moisture levels, grass type, shade amount, and a number of other factors which affect how much watering a system may need. Quite the contrary, modern irrigation systems are set to run for an amount of time with little to no deference to the actual needs of a lawn.

For these reasons, an environmentally friendly system which saves water, saves the environment, reduces impact on water shortages, and utilizes water used at the highest efficiency possible is desired. The present disclosure provides such a system.

Thus, there is a long-felt need in the art for an irrigation system, an irrigation sensor system and smart scheduling for irrigation purposes, processes, and methods of use which improves upon the state of the art and solves a number of problems in the art.

The disclosure herein provides these advantages and others as will become clear from the specification and claims provided.

### SUMMARY OF THE INVENTION

An irrigation system, an irrigation sensor system and smart scheduling for irrigation purposes, processes, and methods of use are presented. The present disclosure relates to an irrigation system, an irrigation sensor system and smart scheduling for irrigation purposes, processes, and methods of use. More specifically, and without limitation, the present disclosure relates to a wireless communication system, a wireless sensor system, and an integrated irrigation controller. More specifically, and without limitation, the present disclosure relates to a plurality of moisture sensors which contribute to irrigation schedules.

Current irrigation systems waste water and/or when using water, water by inefficient means which has not kept up with modern understanding of agricultural science. Modern irrigation systems fail to account for rain, recent rainfall, sun time, soil type, soil moisture levels, grass type, shade amount, and a number of other factors which affect how much watering a system may need. Quite the contrary, modern irrigation systems are set to run for an amount of time with little to no deference to the actual needs of a lawn.

For these reasons, an environmentally friendly system which saves water, saves the environment, reduces impact on water shortages, and utilizes water used at the highest efficiency possible is desired. The present disclosure provides such a system.

Thus, the present disclosure provides solutions and a system which is a long-felt need in the art for an irrigation system, an irrigation sensor system and smart scheduling for irrigation purposes, processes, and methods of use which improves upon the state of the art and solves a number of problems in the art.

Thus, it is a primary object of the disclosure to provide an irrigation system, an irrigation sensor system and smart scheduling for irrigation purposes, processes, and methods of use that improves upon the state of the art.

Another object of the disclosure is to provide an irrigation system, an irrigation sensor system and smart scheduling for irrigation purposes, processes, and methods of use that provides a smart controller.

Yet another object of the disclosure is to provide an irrigation system, an irrigation sensor system and smart scheduling for irrigation purposes, processes, and methods of use that integrates with present systems.

Another object of the disclosure is to provide an irrigation system, an irrigation sensor system and smart scheduling for irrigation purposes, processes, and methods of use that integrates with existing sprinklers and piping.

Yet another object of the disclosure is to provide an irrigation system, an irrigation sensor system and smart scheduling for irrigation purposes, processes, and methods of use that provides a plurality of soil moisture sensors.

Another object of the disclosure is to provide an irrigation system, an irrigation sensor system and smart scheduling for irrigation purposes, processes, and methods of use that provides a plurality of rain sensors.

Yet another object of the disclosure is to provide an irrigation system, an irrigation sensor system and smart scheduling for irrigation purposes, processes, and methods of use that provides a plurality of light sensors.

Another object of the disclosure is to provide an irrigation system, an irrigation sensor system and smart scheduling for irrigation purposes, processes, and methods of use that understands soil types.

Yet another object of the disclosure is to provide an irrigation system, an irrigation sensor system and smart scheduling for irrigation purposes, processes, and methods of use that detects soil type and contents within the soil which affect moisture needed.

Another object of the disclosure is to provide an irrigation system, an irrigation sensor system and smart scheduling for irrigation purposes, processes, and methods of use that senses barometric pressure.

Yet another object of the disclosure is to provide an irrigation system, an irrigation sensor system and smart scheduling for irrigation purposes, processes, and methods of use that senses rain or precipitation.

Another object of the disclosure is to provide an irrigation system, an irrigation sensor system and smart scheduling for irrigation purposes, processes, and methods of use that considers recent weather and future expected weather forecasts for a geographic location.

Yet another object of the disclosure is to provide an irrigation system, an irrigation sensor system and smart scheduling for irrigation purposes, processes, and methods of use that is wireless.

Another object of the disclosure is to provide an irrigation system, an irrigation sensor system and smart scheduling for irrigation purposes, processes, and methods of use that includes a plurality of wireless sensors which communicate with a centralized irrigation controller.

Yet another object of the disclosure is to provide an irrigation system, an irrigation sensor system and smart scheduling for irrigation purposes, processes, and methods of use that can implement a plurality of controllers.

Another object of the disclosure is to provide an irrigation system, an irrigation sensor system and smart scheduling for irrigation purposes, processes, and methods of use that develops smart irrigation schedules for each unique system.

Yet another object of the disclosure is to provide an irrigation system, an irrigation sensor system and smart scheduling for irrigation purposes, processes, and methods of use that continue to adapt and change a schedule over time depending on the circumstances of the system.

Another object of the disclosure is to provide an irrigation system, an irrigation sensor system and smart scheduling for irrigation purposes, processes, and methods of use that utilizes machine learning and artificial intelligence.

Yet another object of the disclosure is to provide an irrigation system, an irrigation sensor system and smart scheduling for irrigation purposes, processes, and methods of use that utilize machine learning and continue to adapt the system for increased efficiencies in irrigation.

Another object of the disclosure is to provide an irrigation system, an irrigation sensor system and smart scheduling for irrigation purposes, processes, and methods of use that are easy to use.

Yet another object of the disclosure is to provide an irrigation system, an irrigation sensor system and smart scheduling for irrigation purposes, processes, and methods of use that are safe to use.

Another object of the disclosure is to provide an irrigation system, an irrigation sensor system and smart scheduling for irrigation purposes, processes, and methods of use that utilizes a global positioning system.

Yet another object of the disclosure is to provide an irrigation system, an irrigation sensor system and smart scheduling for irrigation purposes, processes, and methods of use that provides alerts.

Another object of the disclosure is to provide an irrigation system, an irrigation sensor system and smart scheduling for irrigation purposes, processes, and methods of use that tracks historical data.

Yet another object of the disclosure is to provide an irrigation system, an irrigation sensor system and smart scheduling for irrigation purposes, processes, and methods of use that can run autonomously.

Another object of the disclosure is to provide an irrigation system, an irrigation sensor system and smart scheduling for irrigation purposes, processes, and methods of use that are accurate.

Yet another object of the disclosure is to provide an irrigation system, an irrigation sensor system and smart scheduling for irrigation purposes, processes, and methods of use that works with various digital platforms.

Another object of the disclosure is to provide an irrigation system, an irrigation sensor system and smart scheduling for irrigation purposes, processes, and methods of use that provides a user interface.

Yet another object of the disclosure is to provide an irrigation system, an irrigation sensor system and smart scheduling for irrigation purposes, processes, and methods of use that is quick and efficient.

Another object of the disclosure is to provide an irrigation system, an irrigation sensor system and smart scheduling for irrigation purposes, processes, and methods of use that is easy to program to a predetermined set of rules.

Yet another object of the disclosure is to provide an irrigation system, an irrigation sensor system and smart scheduling for irrigation purposes, processes, and methods of use that are robust.

Another object of the disclosure is to provide an irrigation system, an irrigation sensor system and smart scheduling for irrigation purposes, processes, and methods of use that saves time for a user.

Yet another object of the disclosure is to provide an irrigation system, an irrigation sensor system and smart scheduling for irrigation purposes, processes, and methods of use that saves energy and natural resources.

Another object of the disclosure is to provide an irrigation system, an irrigation sensor system and smart scheduling for irrigation purposes, processes, and methods of use that are high quality.

These and other objects, features, or advantages of the present disclosure will become apparent from the specification and claims.

### BRIEF DESCRIPTION OF DRAWINGS

The drawings accompanying and forming part of this specification are included to depict certain aspects of the disclosure.

FIG. 1 is a top, perspective view of the irrigation system; the view showing a moisture sensor; the view showing the moisture sensor having a top with a dome and dome cover; the view showing a wand extending to the bottom; the view showing the wand having a pointed tip for ease in installation.

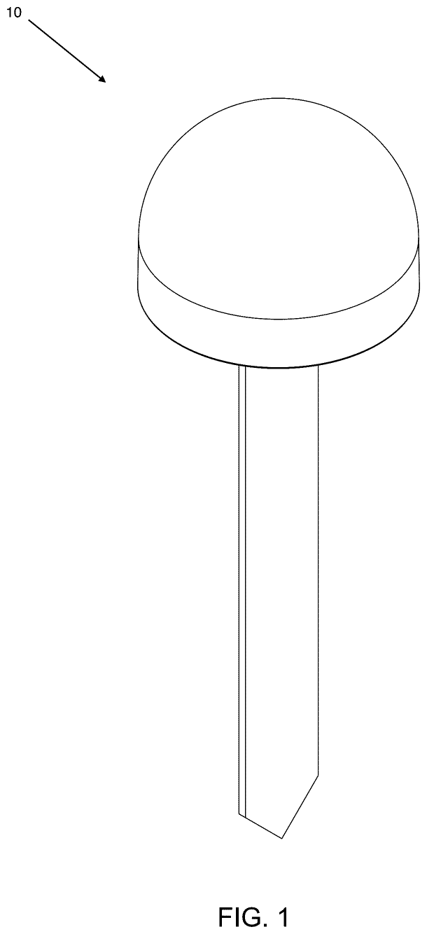

FIG. 2 is a bottom, perspective view of the irrigation system; the view showing a moisture sensor; the view showing the moisture sensor having a top with a dome and dome cover; the view showing a plurality of sensors and attachment features on the bottom of the dome; the view showing a wand extending to the bottom; the view showing the wand having a pointed tip for ease in installation.

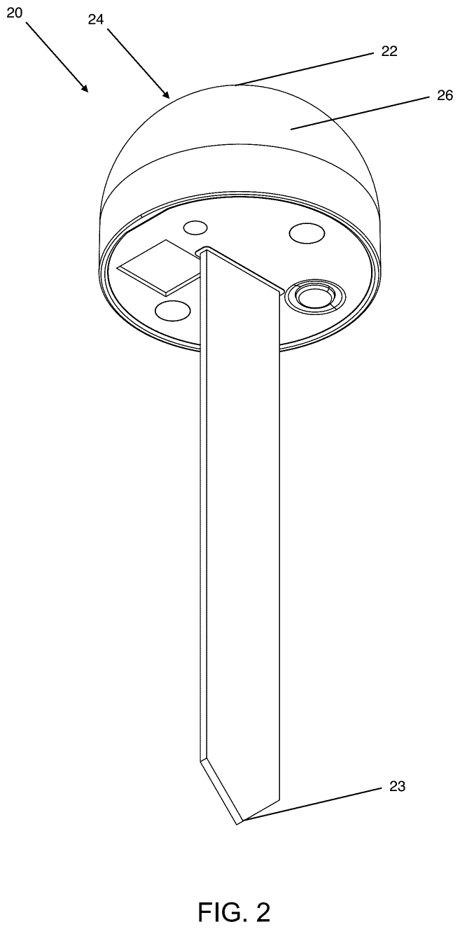

FIG. 3 is a top, isometric perspective view of the irrigation system; the view showing a moisture sensor; the view showing the moisture sensor having a top with a dome and dome cover; the view showing a wand extending to the bottom; the view showing the wand having a pointed tip for ease in installation.

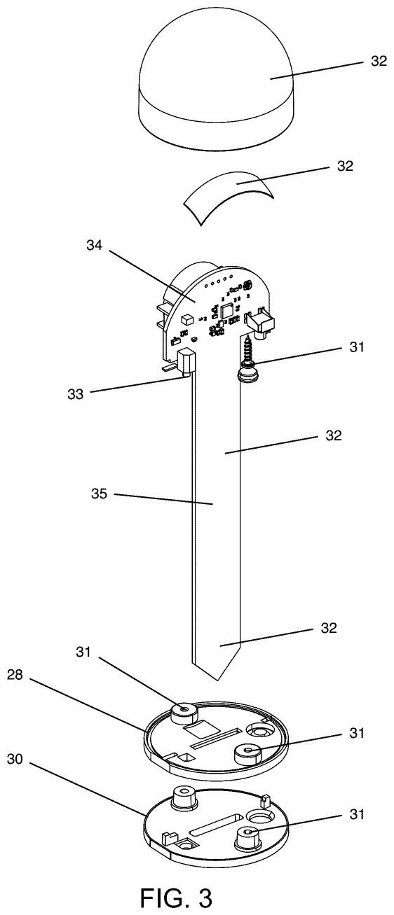

FIG. 4 is a top, isometric perspective view of the irrigation system; the view showing a moisture sensor; the view showing the moisture sensor having a top with a dome and dome cover; the view showing a wand extending to the bottom; the view showing the wand having a pointed tip for ease in installation.

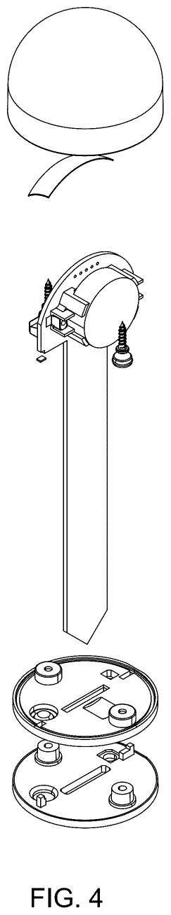

FIG. 5 is a top, perspective view of the irrigation system; the view showing a moisture sensor; the view showing the moisture sensor having a top with a dome and dome cover; the view showing the dome cover removed; the view showing a wand extending to the bottom; the view showing the wand having a pointed tip for ease in installation.

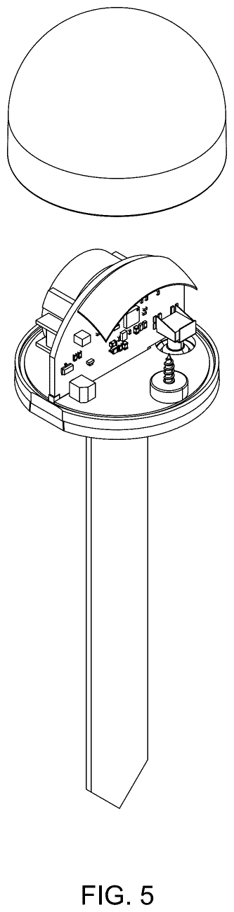

FIG. 6 is a top, perspective view of the irrigation controller; the view showing the irrigation controller in the closed position with the lid closed and engaged with the base; the view showing a plurality of attachment features which operably connect the lid to the base; the view showing a plurality of apertures in the side of the base.

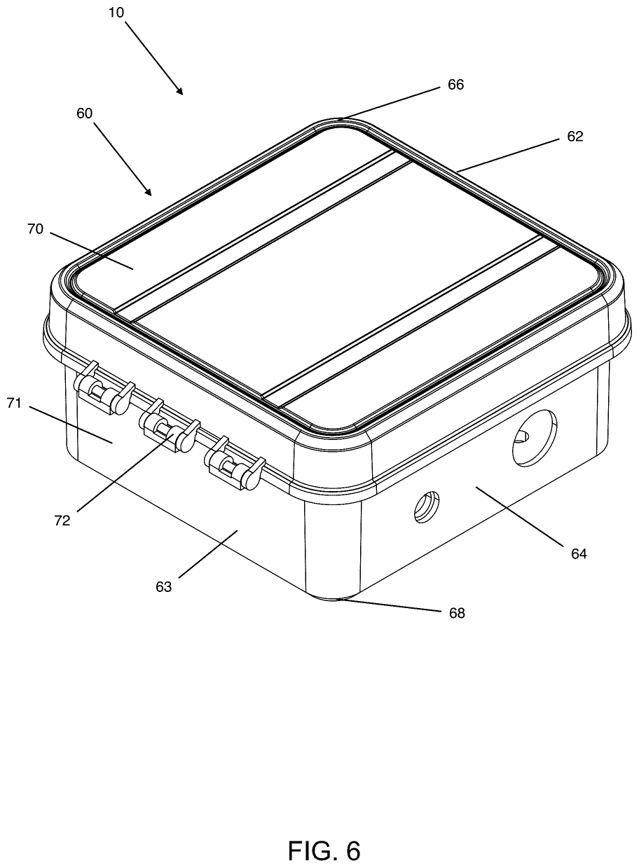

FIG. 7 is a top, perspective view of the irrigation controller; the view showing the irrigation controller in the closed position with the lid closed and engaged with the base; the view showing a clip which provides for connecting the lid to the base; the view showing a plurality of apertures in the side of the base.

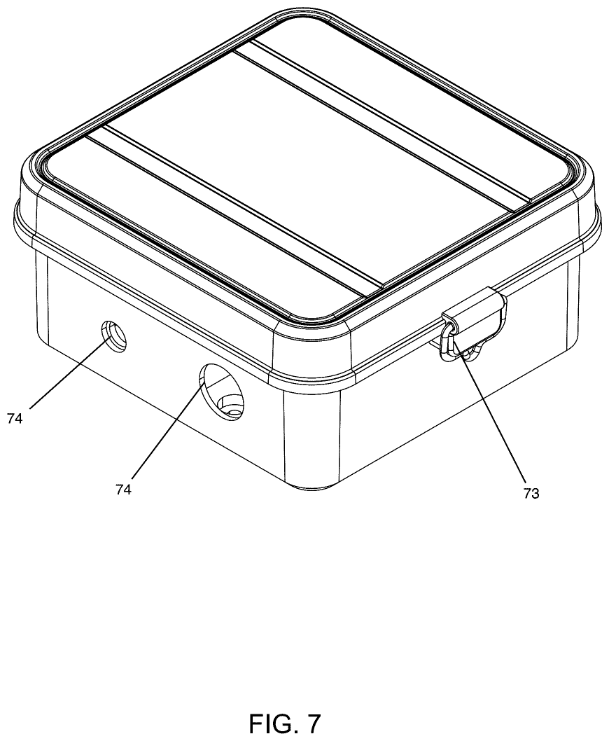

FIG. 8 is a bottom, perspective view of the irrigation controller; the view showing the irrigation controller in the closed position with the lid closed and engaged with the base; the view showing a clip which provides for connecting the lid to the base; the view showing a plurality of apertures in the side of the base; the view showing a plurality of attachment features on the bottom of the base, these attachment features for the purpose of attaching the irrigation controller to a surface; the view showing a plurality of feet on the bottom of the base.

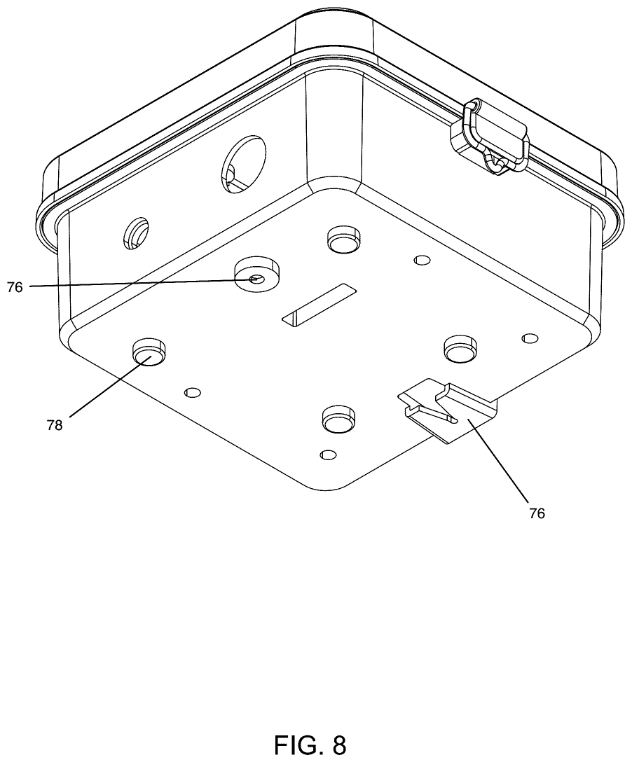

FIG. 9 is a top, perspective view of the irrigation controller; the view showing the irrigation controller in the closed position with the lid open and disengaged with the base; the view showing the hollow interior of the main body of the irrigation controller; the view showing a power supply; the view showing a plurality of zone connections.

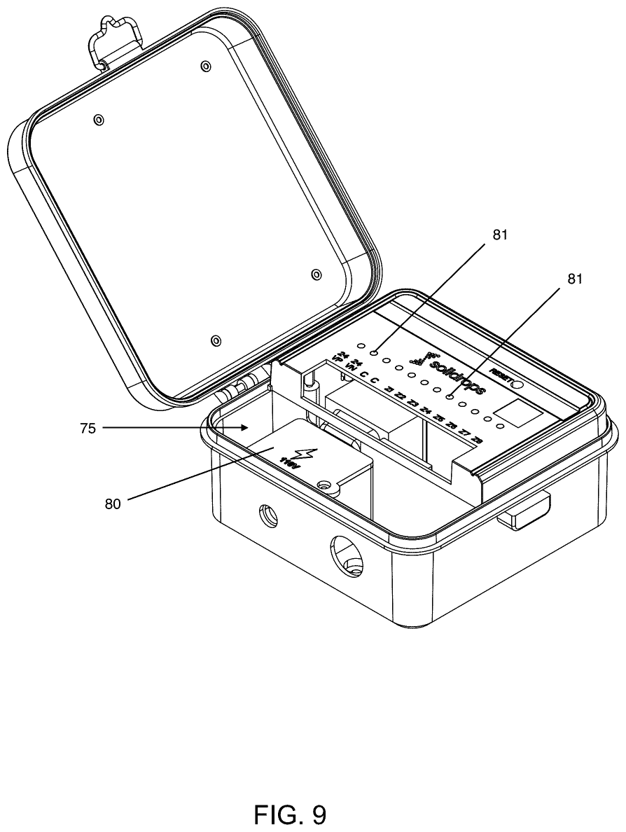

FIG. 10 is a top, isometric view of the irrigation controller; the view showing the irrigation controller in the closed position with the lid open and disengaged with the base; the view showing the hollow interior of the main body of the irrigation controller; the view showing a power supply; the view showing a plurality of zone connections.

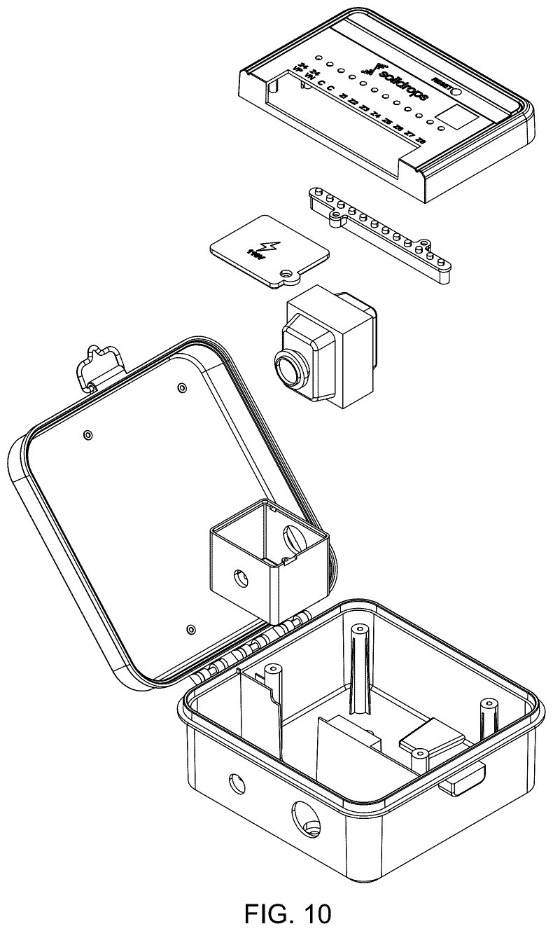

FIG. 11 is a top, perspective view of one embodiment of a signal repeater; the view showing the signal repeater main body extending a length from a first end to a second end between opposing sides and a top and a bottom; the view showing a power supply.

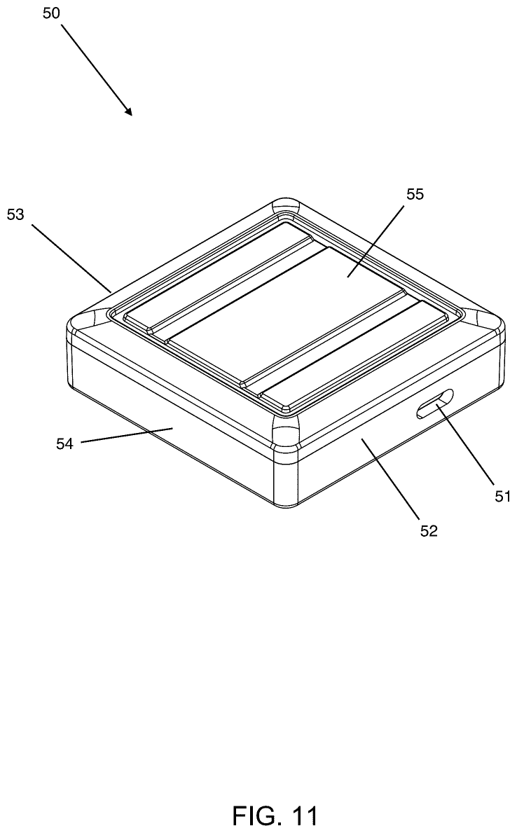

FIG. 12 is a top, perspective view of one embodiment of a signal repeater; the view showing the signal repeater main body extending a length from a first end to a second end between opposing sides and a top and a bottom.

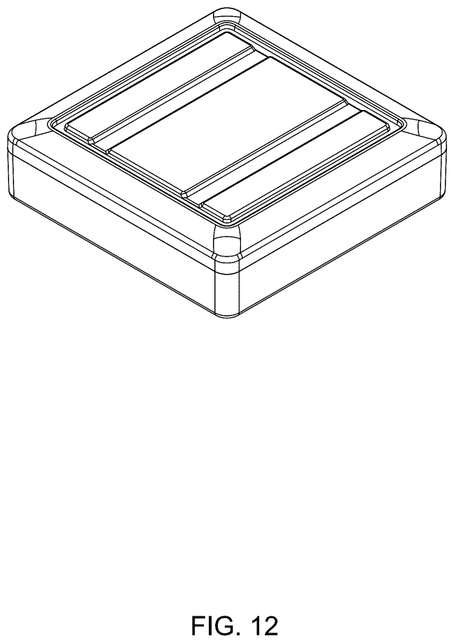

FIG. 13 is a top, perspective view of one embodiment of a signal repeater; the view showing the signal repeater main body extending a length from a first end to a second end between opposing sides and a top and a bottom; the view showing a hollow interior with a PCB.

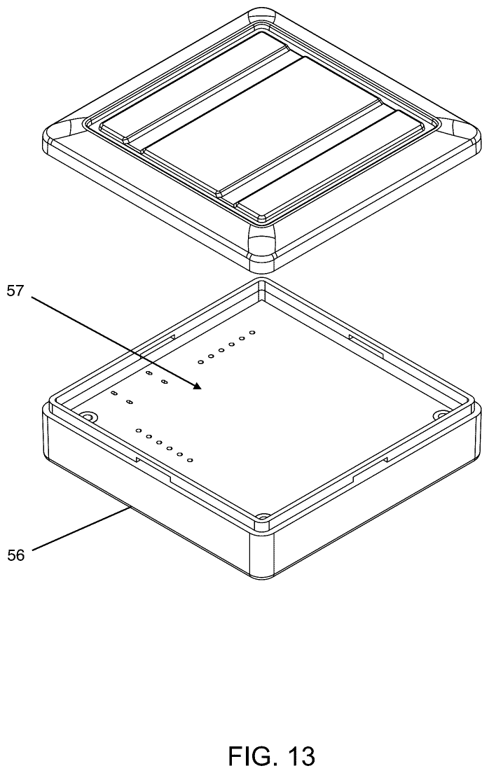

FIG. 14 is a top, perspective view of one embodiment of a signal repeater; the view showing the signal repeater main body extending a length from a first end to a second end between opposing sides and a top and a bottom; the view showing a hollow interior with a PCB.

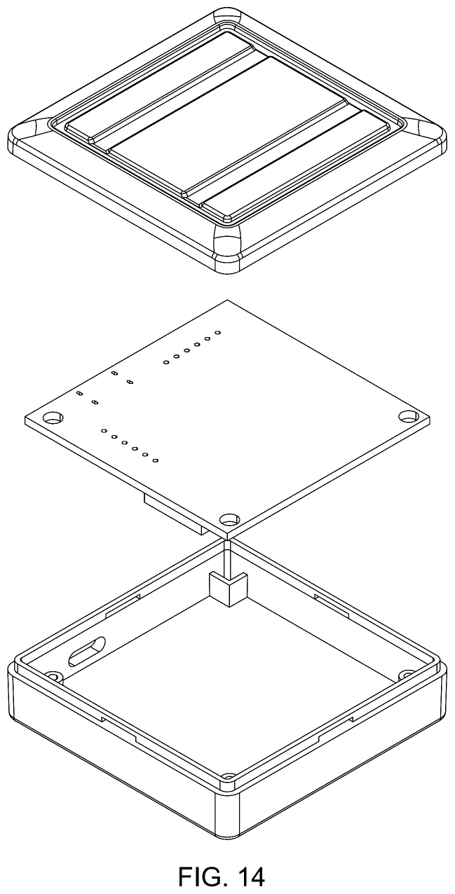

FIG. 15 is a bottom, perspective view of one embodiment of a signal repeater; the view showing the signal repeater main body extending a length from a first end to a second end between opposing sides and a top and a bottom; the view showing a hollow interior with a PCB.

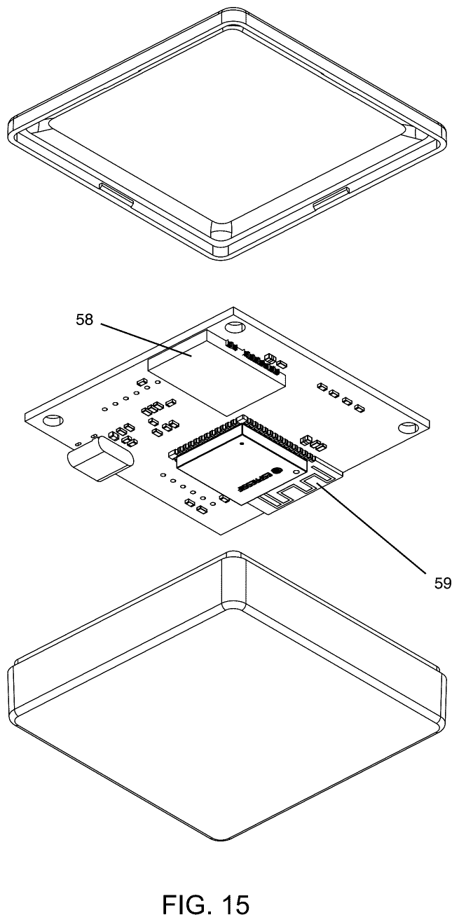

FIG. 16 is a diagram illustrating various components of the system and interaction of some of these components.

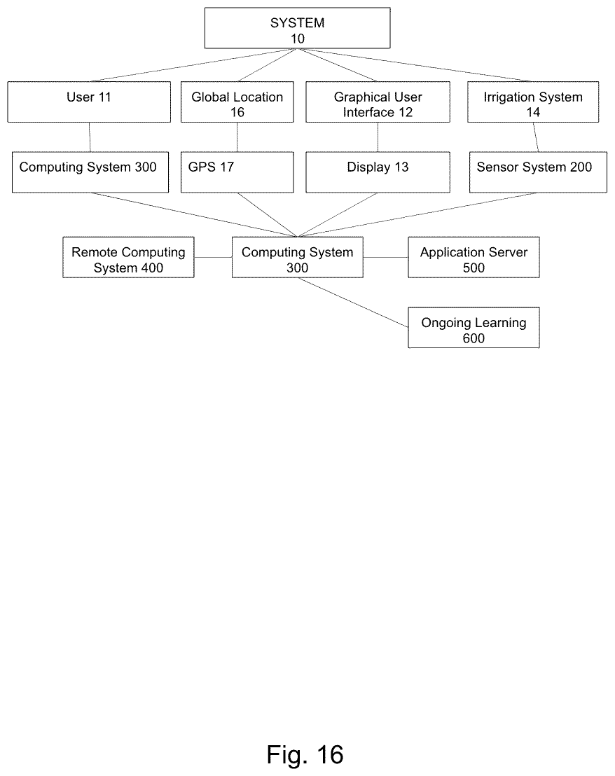

FIG. 17 is a diagram illustrating ongoing learning interactions of system 10.

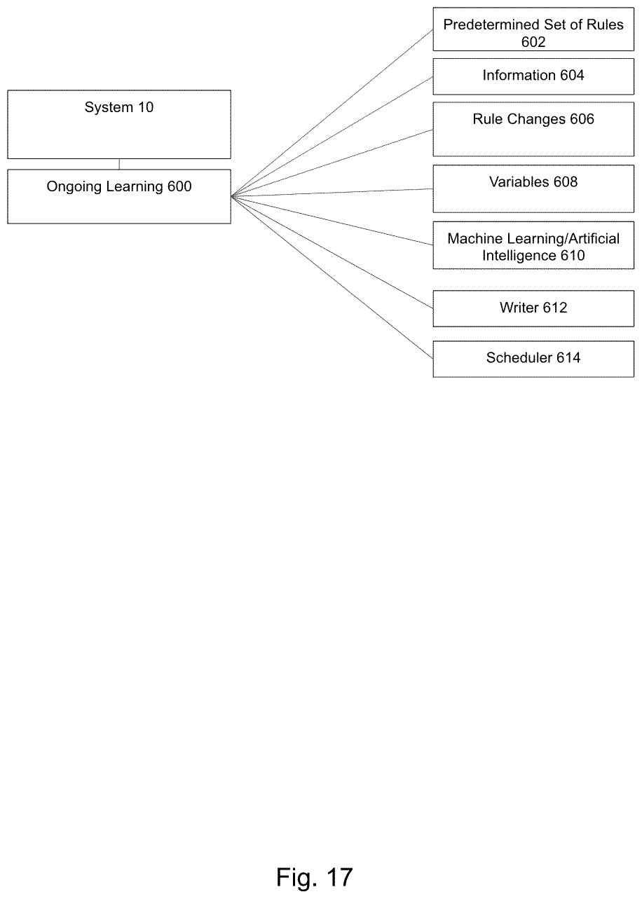

FIG. 18 is a diagram illustrating the computing system and some of the components of the computing system in one example of one embodiment.

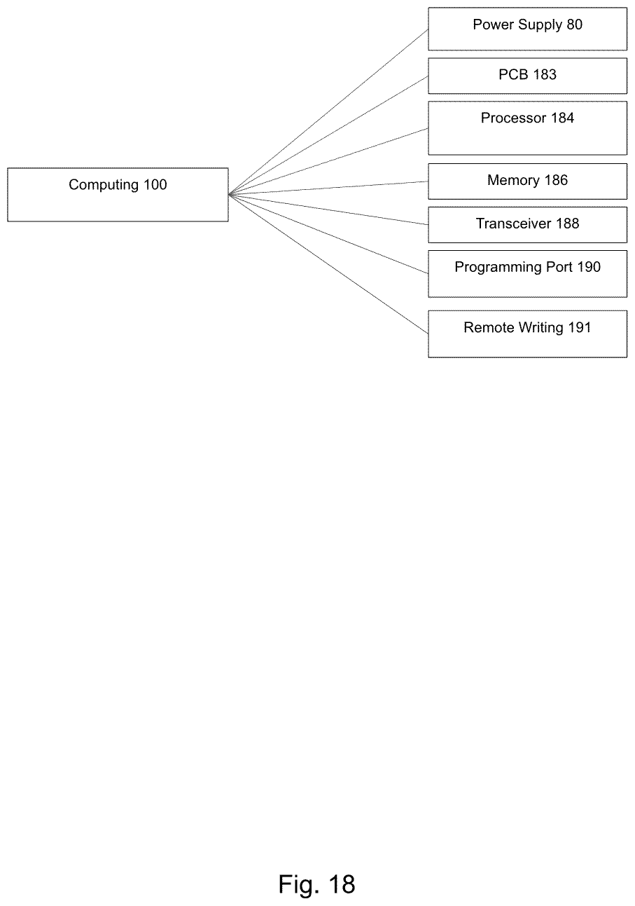

### DESCRIPTION

In the following detailed description, reference is made to the accompanying drawings which form a part hereof, and in which is shown by way of illustration specific embodiments in which the disclosure may be practiced. These embodiments are described in sufficient detail to enable those skilled in the art to practice the disclosure, and it is to be understood that other embodiments may be utilized and that mechanical, procedural, and other changes may be made without departing from the spirit and scope of the disclosure(s). The following detailed description is, therefore, not to be taken in a limiting sense, and the scope of the disclosure(s) is defined only by the appended claims, along with the full scope of equivalents to which such claims are entitled.

As used herein, the terminology such as vertical, horizontal, top, bottom, front, back, end, sides and the like are referenced according to the views, pieces and figures presented. It should be understood, however, that the terms are used only for purposes of description, and are not intended to be used as limitations. Accordingly, orientation of an object or a combination of objects may change without departing from the scope of the disclosure.

Reference throughout this specification to “one embodiment,” “an embodiment,” “one example,” or “an example” means that a particular feature, structure, or characteristic described in connection with the embodiment or example is included in at least one embodiment of the present disclosure. Thus, the appearance of the phrases “in one embodiment,” “in an embodiment,” “one example,” or “an example” in various places throughout this specification are not necessarily all referring to the same embodiment or example. Furthermore, the particular features, structures, databases, or characteristics may be combined in any suitable combinations and/or sub-combinations in one or more embodiments or examples. In addition, it should be appreciated that the figures provided herewith are for explanation purposes to persons ordinarily skilled in the art and that the drawings are not necessarily drawn to scale.

Embodiments in accordance with the present disclosure may be embodied as an apparatus, method, or computer program product. Accordingly, the present disclosure may take the form of an entirely hardware-comprised embodiment, an entirely software-comprised embodiment (including firmware, resident software, micro-code, etc.), or an embodiment combining software and hardware aspects that may all generally be referred to herein as a “circuit,” “module,” or “system.” Furthermore, embodiments of the present disclosure may take the form of a computer program product embodied in any tangible medium.

Any combination of one or more computer-usable or computer-readable media may be utilized. For example, a computer-readable medium may include one or more of a portable computer removable drive, a hard disk, a random access memory (RAM) device, a read-only memory (ROM) device, an erasable programmable read-only memory (EPROM or Flash memory) device, a portable compact disc read-only memory (CDROM), an optical storage device, and a magnetic storage device. Computer program code for carrying out operations of the present disclosure may be written in any combination of one or more programming languages. Such code may be compiled from source code to computer-readable assembly language or machine code, or virtual code, or framework code suitable for the disclosure herein, or machine code suitable for the device or computer on which the code will be executed.

Embodiments may also be implemented in cloud computing environments. In this description and the following claims, “cloud computing” may be defined as a model for enabling ubiquitous, convenient, on-demand network access to a shared pool of configurable computing resources (e.g., networks, servers, storage, applications, and services) that can be rapidly provisioned via virtualization and released with minimal management effort or service provider interaction and then scaled accordingly. A cloud model can be composed of various characteristics (e.g., on-demand self-service, broad network access, resource pooling, rapid elasticity, and measured service), service models (e.g., Software as a Service (“Saas”), Platform as a Service (“PaaS”), and Infrastructure as a Service (“IaaS”)), and deployment models (e.g., private cloud, community cloud, public cloud, and hybrid cloud).

The flowchart and block diagrams in the attached figures illustrate the architecture, functionality, and operation of possible implementations of systems, methods, and computer program products according to various embodiments of the present disclosure. In this regard, each block in the flowchart or block diagrams may represent a module, segment, or portion of code, which comprises one or more executable instructions for implementing the specified logical function(s). It will also be noted that each block of the block diagrams and/or flowchart illustrations, and combinations of blocks in the block diagrams and/or flowchart illustrations, may be implemented by special purpose hardware-based systems that perform the specified functions or acts, or combinations of special purpose hardware and computer instructions. These computer program instructions may also be stored in a computer-readable medium that can direct a computer or other programmable data processing apparatus to function in a particular manner, such that the instructions stored in the computer-readable medium produce an article of manufacture including instruction means which implement the function/act specified in the flowchart and/or block diagram block or blocks.

### System:

With reference to the figures, an irrigation system, an irrigation sensor system and smart scheduling for irrigation purposes, processes, and methods of use 10 are presented. an irrigation system, an irrigation sensor system and smart scheduling for irrigation purposes, processes, and methods of use 10 (hereafter referred to as “irrigation system”, “wireless irrigation system”, “communication system”, “irrigation controller system”, or simply “system”) is formed of any suitable size, shape and design.

In the arrangement shown, as one example, the irrigation system is configured with a plurality of wireless soil moisture sensors and a plurality of irrigation controllers. The system utilizes a predetermined set of rules and ongoing machine learning to adapt the rules. The system also integrates artificial intelligence and a number of other components to automatically provide irrigation to a desired system which saves water, energy, time, and provides benefits to a user and a global community.

In the arrangement shown, as one example, system 10 includes at least one moisture sensor 20, an irrigation system, an irrigation controller 60 or headquarters 60, and a computing platform 300.

In the arrangement shown, as one example, system 10 may comprise remote servers, databases, application servers, application databases, product databases, mobile applications, and/or computers; all of which in continuity or as separate acts fulfill the functions disclosed herein.

System 10 also includes, in the embodiment(s) depicted, a graphical user interface, a user, a sensor system 200, a computing system 300, a communication and/or control system 400, an application server, and a mobile computing application, among other components, features, and functions.

### User (or Plurality Thereof):

In the arrangement shown, as one example, system 10 includes a user. User may be any user interacting with or utilizing the system 10. This may include viewing, controlling, analyzing, manipulating, and/or interacting with system 10. User is not limited to a single user but may be a plurality of users.

### Irrigation Controller:

In the arrangement shown, as one example, system 10 includes at least one irrigation controller (also referred to as “controller”). Controller is formed of any suitable size, shape, and design, and is configured to operate automatic irrigation systems such as lawn sprinklers and drip irrigation systems, and the like. In the arrangement shown, the irrigation controller opens and closes valves which deliver water to a particular part and/or to all of the system. The irrigation controller also accepts information from moisture sensors, the internet, national weather services, and through other sources to inform decisions and learn to make improved irrigation decisions.

In the arrangement shown, as one example, the irrigation controller 60 is formed generally of a box with a base 71 and a lid 70. The irrigation controller 60 extends a length from a first end 62 to a second end 63—between opposing sides. The irrigation controller 60 includes a top 66 and a bottom 68—having a lid 70 forming the top portion and a base 71 forming a lower portion. The irrigation controller 60 includes a plurality of attachment features 72 for securing the lid to the base. The controller 60 also includes a clip 73 for closing the lid 70.

The irrigation controller 60 also includes a plurality of apertures 74 for providing for wiring to enter the hollow interior 75 of the controller. The irrigation controller 60 also includes a plurality of attachment feature 76 for securing to a location such as a vertical wall or ceiling, or the like. The irrigation controller 60 also includes a plurality of feet 78 so that the controller 60 can engage a surface such as a ground surface, wall surface, table surface, or the like.

Furthermore, and in the arrangement shown, irrigation controller 60 includes a power supply 80, a plurality of zone connections 81, an onboard computing platform.

### Plurality of Moisture Sensors:

In the arrangement shown, as one example, system 10 includes a plurality of moisture sensors 20. Plurality of moisture sensors 20 are formed of any suitable size, shape, and design, and is configured to detect moisture in soil at varying levels and/or depts, to detect moisture on the surface, and relay this information wirelessly to an irrigation controller and/or controllers for use in determining when and where to deliver additional water.

In the arrangement shown, as one example, plurality of moisture sensors 20 or moisture sensor 20, or simply sensor 20 extends a length from a top 22 to a bottom 23 with a dome 24, a cover 26, a first plate 28, a second plate 30, a plurality of attachment features 31, a plurality of sensors 32 (see sensor system herein), a plurality of indicators 33, a PCB 24, and a wand 35—wand extending into the soil surface having a pointed end nearest the bottom for easily sliding into a soil surface and for sensing moisture levels. In some embodiments multiple moisture sensors are included in the wand along the length for detecting soil moisture levels at different depths within the soil.

### Signal Repeater:

In the arrangement shown, as one example, system 10 includes a signal repeater 50. Signal repeater (or “ebyte module” or “module”) is formed of any suitable size, shape, and design. In one example, the signal repeater is a relative box like in shape. This signal repeater serves to send and/or receive signals. Similarly, this signal repeater serves to transmit signals and/or boost signals so that the various components of system 10 such as a plurality of moisture sensors placed throughout an irrigation system can effectively and efficiently communicate with the irrigation controller 60 and/or other moisture sensors.

In the arrangement shown, as one example, signal repeater 50 extends a length from a first end 52 to a second end 53 having opposing sides 54 and a top 55 and a bottom 56. In the arrangement shown, Signal repeater 50 includes onboard computing and processing housed in a hollow interior and may also include a plurality of antennae and transceivers, and a power supply 51.

### Ongoing Rule Learning:

In the arrangement shown, as one example, system 10 includes a scheduler 600. Scheduler is configured to operate under a predetermined set of rules 602 which adds information 604 and adopts rule changes 606 to deliver moisture and/or activating parts of a system dependent on a number of variables 608, such as weather in the region, soil moisture levels, expected rainfall, soil type, grass type, vegetation type, desired lawn appearance, draught ratings, satellite data, plant database information, and more. Schedulers may incorporate machine learning and artificial intelligence 610. Scheduler 600 includes a writer 612 and a rule 614. The writer re-rewrites the rule 614 at anticipated and/or programmed timeframes and/or under certain conditions and/or changes in the condition such that watering scheduling will change based on the changes implemented or written by the writer to the scheduler. In this way, the present system can understand unique water needs and optimize water usage efficiency for various applications.

### Irrigation System:

In the arrangement shown, as one example, system 10 includes an irrigation system 14. Irrigation system 14 is formed of any suitable size, shape, and design, and is configured to deliver moisture and/or watering to a lawn, landscape, garden, crop, and the like. Irrigation system may be formed of a single zone in a single set of pipes or may be linked to a plurality of zones over several different systems. Irrigation system may be above ground and/or below ground systems including pipes, sprinklers, connections, pumps, nozzles, wires, electronics, a combination thereof, and the like. The irrigation system is located somewhere on earth and includes a global location 16 accessed via the internet, bluetooth from a mobile device with GPS 17, may have GPS built in, or the like.

### Smart Device:

In an alternative embodiment, system 10 may also include a smart device. A smart device may be a smart phone, a laptop, a tablet, or other connected devices and the like. The smart device may serve as the display for the graphical user interface.

### Power Supply:

Various components of system 10, such as the irrigation controller, the signal repeater, and the moisture sensor include a power supply. This may be a hardwired power supply, a plug, a solar power supply, a plurality of batteries, a plurality of rechargeable batteries. In one arrangement, the onboard batteries have been configured to last three years or more.

### Graphical User Interface:

In the arrangement shown, as one example, system 10 may include a graphical user interface. Graphical user interface 12 is formed of any suitable size shape and design and is configured to allow a user to view interact with, manipulate, and visually access system data and information, information related thereto, and/or view various data for various environments and/or add information to system and/or environment and/or change the settings of the sensors and/or change the settings of operation.

Graphical user interface 12 is an exemplary method by which systems of the present disclosure may operate and/or make programming changes to the operation of system 10. Employing graphical user interface 12, enhances a user's interactions with system 10 in the form of awareness and knowledge of data within the system 10. System 10 provides direct links to a user's history, settings, programming, and other information related to a particular function and/or a particular component and/or a particular user and/or a particular operation. In this way graphical user interface 12 provides for a means for a user to make adjustments to the operation and functionality of system 10.

Display of Graphical User Interface: In the arrangement shown, as one example, system 10 includes a graphical user interface 12. Graphical user interface 12 may include a display 13, which is configured to show and display information, including data, for review and interpretation by a user or plurality of users, or a plurality of users interacting with one another.

In the arrangement shown, as one example, interactive user display is formed of a display screen, such as that of a desktop computer, laptop computer, monitor, tablet, smart phone, smart TV, projector, virtual reality display or any other device or form of a display. In the arrangement shown, as one example, interactive user display comprises a series of interactive user display pages, however, the interactive user display may consist of a single page or any other method of displaying information on a display as could be adapted to various size screens, devices, or user preferences. The interactive user display can display various information and/or functional information which is retrieved and/or requested.

### Computing Platform:

In the arrangement shown, as one example, system 10 includes a computing platform 300 (or “computer”, or “computer platform”). Computing platform 300 is formed of any suitable size, shape, and design and is configured to provide computing support, power, and computing processing for both onboard computing functionality as well as communication for off-board or server computing functionality. In this way, an onboard computing system, among other components and features on top of the platform.

In the arrangement shown, as one example, system 10 includes a computer 300. Computer 300 is formed of any suitable size, shape, and design and is configured to provide for the main off-board computing processing and implementation of computer handling of data from data gathering performed.

### Onboard Computing System:

In one arrangement, as is shown, system 10 includes an onboard computing system (or “onboard computing device”). Onboarding computing system is formed of any suitable size, shape, and design and configured to handle onboard computing operations, as are necessary for the operation of system 10. Onboarding computing device is connected with electronic network and/or database and/or server or cloud via communication means, bluetooth communication, bluetooth low energy chip (BLE onboard), and may include a processor, a memory, a microcontroller, a printed circuit board, a microprocessor, a receiver/transceiver, may include at least one antenna, and a global positioning system, among other components.

In the arrangement shown, as one example, onboard computing system 100 includes an onboard PCB 183, and onboard processor 184, an onboard memory 186, and an onboard transceiver 188, a programming port 190, a remote writing system 191 implementing artificial intelligence.

Computing device may be formed of and/or include any computing device capable of displaying and manipulating data in the manners described herein. Computing device may include for example a desktop computer, a laptop computer, a tablet, smart phone, or any other computing device or other interactive device.

### Remote Computing System:

In one arrangement, as is shown, system 10 includes a remote computing system 400 (or “remote computing device”). Remote computing devices 400 are formed of any suitable size, shape, and design and configured to handle onboard computing operations, as are necessary for the operation of system 10. Remote computing device is connected with electronic network and/or database and/or server or cloud via communication means and includes a processor, a memory, a microcontroller, a printed circuit board, a microprocessor, a receiver/transceiver, may include at least one antenna, a power supply, and a communications system, among other components.

### Sensor System (Moisture Sensing):

In the arrangement shown, as one example, system 10 includes a sensor system 200. Moisture sensors are designed to be located within the soil and/or around a yard and/or lawn and/or area of irrigation. The moistures are designed and configured to sense moisture levels are various depths of soil and/or the surface and/or detect rain and/or detect humidity levels, barometric levels, sun levels and/or light levels and more. Moisture sensor system is formed of any suitable size, shape, and design and may include one or more sensors and/or one or more sensing technologies. In the arrangement shown, as one example, the sensor system is configured to detect and communicate information related to system 10 as well as the surroundings and/or environment of system 10.

In the arrangement shown, as one example, various sensors are utilized within system 10 to detect system status such as distance, temperature changes, and other operating status parameters within system 10.

In the arrangement shown, as one example, a sensory system and perhaps a LIDAR sensor system is utilized to understand the environment surrounding system 10 is engaging with. This might include both distance and imaging sensors and/or camera sensors.

### Other Sensors:

In addition to the distance and image sensors discussed herein, system 10 may also include other sensors, such as temperature sensors, moisture sensors, heat sensors, light sensors, motion sensors, and other sensors. In the arrangement shown, as one example, system 10 includes at least one other sensor. Other sensors are formed of any suitable size, shape and design and are configured to facilitate sensing of surfaces and/or environmental information and converting the characteristics of the outside space and/or environment into computer readable information. Other sensors are used to detect and respond to some type of input from the physical environment.

Other sensors may be used for sensing a single component of an environment. For example, the specific input of other sensors may be light, heat, motion, moisture, pressure, or any one of a great number of other information related to system 10. Another sensor is a device, module, or subsystem whose purpose is to detect events or changes in system 10 and send the information to other electronics, frequently a computer processor. The output of another sensor is generally a signal that is generally converted to human-readable display at the sensor location or transmitted electronically over a network for reading or further processing. Both analog sensors and/or digital sensors are hereby contemplated for use. In one arrangement, another sensor and/or microsensor sends information to a processor for use with other electronics.

### Application Server:

In the arrangement shown, as one example, system 10 may comprise remote servers, databases, and/or computers that fulfill the functions disclosed and described herein. In the embodiment depicted, system 10 comprises an application server 500. Application server 500 comprises one or more computer systems adapted to transmit and receive data regarding selected datasets related to various users and/or datasets related to multiple users. Application server 500 is adapted to query databases, and may utilize unique identification codes, to retrieve information and associated information related to system 10.

In addition to the above identified features, options, controls, and components, system 10 may also include other features and functionalities, among other options, controls, and components.

It will be appreciated by those skilled in the art that other various modifications could be made to the system, process, and method of use without parting from the spirit and scope of this disclosure. All such modifications and changes fall within the scope of the claims and are intended to be covered thereby.

1. An irrigation system, comprising:
a moisture sensor;
the moisture sensor extending a length from a top to a bottom;
the moisture sensor having a dome;
the moisture sensor having a first plate;
the moisture sensor having a plurality of sensors;
the moisture sensor having a wand;
the wand having a pointed end for ease of installation; wherein the pointed end is configured to provide ease of installation for driving the moisture sensor into soil at a depth;
the moisture sensor having a power supply;
an irrigation controller;
the irrigation controller extending a length from a first end to a second end between opposing sides;
the irrigation controller having a top and a bottom forming a hollow interior;
the irrigation controller having a power supply;
the irrigation controller having a plurality of zone connections;
the irrigation controller having a programming port;
wherein the moisture sensor detects a moisture level in a soil and relays the moisture level to the irrigation controller; wherein the irrigation controller makes adjustments to an irrigation schedule based on the moisture level in the soil.

2. The system of claim 1, further comprising:
an irrigation system;
wherein the irrigation system is configured to deliver water to a landscape;
the irrigation system having a global location;
wherein the global location is configured through a wireless connection.

3. The system of claim 1, further comprising:
a global positioning system.

4. The system of claim 1, further comprising:
the moisture sensor having an onboard computing system;
the moisture sensor having an onboard processor;
the moisture sensor having an onboard memory;
the moisture sensor having an onboard transceiver.

5. The system of claim 1, further comprising:
a plurality of batteries.

6. The system of claim 1, further comprising:
a plurality of batteries; wherein the plurality of batteries are rechargeable batteries.

7. The system of claim 1, further comprising:
a user.

8. The system of claim 1, further comprising:
a graphical user interface;
the graphical user interface having a display;
wherein the display of the graphical user interface is a smart device.

9. The system of claim 1, further comprising:
the moisture sensor having a cover;
the moisture sensor having a second plate;
the moisture sensor having a plurality of attachment features.

10. The system of claim 1, further comprising:
the moisture sensor having a plurality of indicators;
wherein the plurality of indicators are formed of light emitting diodes connected to a PCB.

11. The system of claim 1, further comprising:
wherein the wand is configured with the plurality of sensors at varying depths from a top to a bottom of the wand; such that the plurality of sensors can detect moisture levels at varying depths of soil.

12. The system of claim 1, further comprising:
the signal repeater having an onboard computing system;
the signal repeater having a plurality of antennae.

13. The system of claim 1, further comprising:
a signal repeater;
the signal repeater extending a length from a first end to a second end between opposing sides;
the signal repeater having a top and a bottom forming a hollow interior;
the signal repeater having at least one transceiver;
wherein the signal repeater is configured to intake signals from the plurality of sensors;
wherein the signal repeater is configured to repeat signals from the plurality of sensors; wherein repeating is sending signals from the plurality of sensors;
the signal repeater having a power supply.

14. The system of claim 1, further comprising:
the irrigation controller having a lid;
the irrigation controller having a base;
the irrigation controller having a plurality of attachment features; wherein the plurality of attachment features are operably connected to the lid and to the base such that the lid can open and close relative to the base;
the irrigation controller having a clip; wherein the clip is configured to hold the lid in a closed position relative to the base and allow the lid to open relative to the base;
the irrigation controller having a plurality of apertures;
the irrigation controller having a plurality of attachment points of the bottom of the base; wherein the plurality of attachment points are configured to attach the irrigation controller to a surface;
the irrigation controller having a plurality of feet.

15. The system of claim 1, further comprising:
a computing platform;
a remote computing platform;
a sensor system;
an application server.

16. The system of claim 1, further comprising:
an ongoing learning system;
the ongoing learning system having a predetermined set of rules;
the ongoing learning system having a database; wherein the database is configured to accept information;
the ongoing learning system having rule changes;
the ongoing learning system having a plurality of variables;
the ongoing learning system having machine learning.

17. The system of claim 1, further comprising:
an ongoing learning system;
the ongoing learning system having a writer;
the ongoing learning system having a scheduler;
wherein the writer receives an input based on processing completed by the ongoing learning system; wherein the writer adjusts the scheduler at predetermined intervals of time; wherein the scheduler receives the writing from the writer and schedules irrigation activities for durations of time at particular points in time.

18. A smart scheduling and sensing system for irrigation, comprising:
an irrigation system;
wherein the irrigation system is configured to deliver water to a landscape;
a global location;
wherein the global location is configured through a wireless connection;
a global positioning system;
a plurality of moisture sensors;
the plurality of moisture sensors each extending a length from a top to a bottom;
the plurality of moisture sensors each having a dome;
the plurality of moisture sensors each having a cover;
the plurality of moisture sensors each having a first plate;
the plurality of moisture sensors each having a second plate;
the plurality of moisture sensors each having a plurality of attachment features;
the plurality of moisture sensors each having a plurality of sensors;
the plurality of moisture sensors each having a plurality of indicators;
wherein the plurality of indicators are formed of light emitting diodes connected to a PCB;
the plurality of moisture sensors each having a wand;
the wand having a pointed end for ease of installation; wherein the pointed end is configured to provide ease of installation for driving the plurality of moisture sensors into soil at a depth;
wherein the wand is configured with the plurality of sensors at varying depths from a top to a bottom of the wand; such that the plurality of sensors can detect moisture levels at varying depths of soil;
the plurality of moisture sensors having an onboard computing system;
the plurality of moisture sensors having an onboard processor;
the plurality of moisture sensors having an onboard memory;
the plurality of moisture sensors having an onboard transceiver;
the plurality of moisture sensors having a power supply;
a signal repeater;
the signal repeater extending a length from a first end to a second end between opposing sides;
the signal repeater having a top and a bottom forming a hollow interior;
the signal repeater having an onboard computing system;
the signal repeater having a plurality of antennae;
the signal repeater having at least one transceiver;
wherein the signal repeater is configured to intake signals from the plurality of sensors;
wherein the signal repeater is configured to repeat signals from the plurality of sensors; wherein repeating is sending signals from the plurality of sensors;
the signal repeater having a power supply;
an irrigation controller;
the irrigation controller extending a length from a first end to a second end between opposing sides;
the irrigation controller having a top and a bottom forming a hollow interior;
the irrigation controller having a lid;
the irrigation controller having a base;
the irrigation controller having a plurality of attachment features; wherein the plurality of attachment features are operably connected to the lid and to the base such that the lid can open and close relative to the base;
the irrigation controller having a clip; wherein the clip is configured to hold the lid in a closed position relative to the base and allow the lid to open relative to the base;
the irrigation controller having a plurality of apertures;
the irrigation controller having a plurality of attachment points of the bottom of the base; wherein the plurality of attachment points are configured to attach the irrigation controller to a surface;
the irrigation controller having a plurality of feet;
the irrigation controller having a power supply;
the irrigation controller having a plurality of zone connections;
the irrigation controller having a programming port;
a computing platform;
a remote computing platform;
an ongoing learning system;
the ongoing learning system having a predetermined set of rules;
the ongoing learning system having a database; wherein the database is configured to accept information;
the ongoing learning system having rule changes;
the ongoing learning system having a plurality of variables; the variables providing input for the writer in changing the schedule of the scheduler;
the ongoing learning system having machine learning;
the ongoing learning system having a writer;
the ongoing learning system having a scheduler;
wherein the writer receives an input based on processing completed by the ongoing learning system; wherein the writer adjusts the scheduler at predetermined intervals of time;
wherein the scheduler receives the writing from the writer and schedules irrigation activities for durations of time at particular points in time.

19. The system of claim 18, further comprising:
a graphical user interface;
the graphical user interface having a display;
wherein the display of the graphical user interface is a smart device;
a sensor system;
an application server.

20. A method of lawn irrigation which utilizes sensors and machine learning, comprising the steps:
providing an ongoing learning system, the ongoing learning system having a predetermined set of rules; the ongoing learning system having a database; wherein the database is configured to accept information; the ongoing learning system having rule changes; the ongoing learning system having a plurality of variables; the ongoing learning system having machine learning;
providing a writer;
providing a scheduler; wherein the writer receives an input based on processing completed by the ongoing learning system; wherein the writer adjusts the scheduler at predetermined intervals of time; wherein the scheduler receives the writing from the writer and schedules irrigation activities for durations of time at particular points in time.
